SEQUENCE LISTING (10 sequences)

SEQ ID NO: 1 - protein, 13 aa, Artificial
  Corresponds to a portion of human MEKK3
YSDGRRTFPRIRR

SEQ ID NO: 2 - protein, 13 aa, Artificial
  Corresponds to a portion of human MEKK2
YNDGRKTFPRARR

SEQ ID NO: 3 - protein, 5 aa, Artificial
  Corresponds to a portion of human MEKK
RXTXP

SEQ ID NO: 4 - protein, 5 aa, Artificial
  Corresponds to a portion of human MEKK
RXSXP

SEQ ID NO: 5 - protein, 4 aa, Artificial
  Corresponds to a portion of human MEKK
TFPR

SEQ ID NO: 6 - protein, 5 aa, Artificial
  Corresponds to a portion of human MEKK3
RRTFP

SEQ ID NO: 7 - protein, 5 aa, Artificial
  Corresponds to a portion of human MEKK2
RKTFP

SEQ ID NO: 8 - protein, 6 aa, Artificial
  Corresponds to a portion of human MEKK2
GRKTFP

SEQ ID NO: 9 - protein, 6 aa, Artificial
  Corresponds to a portion of human MEKK3
GRRTFP

SEQ ID NO: 10 - protein, 10 aa, Artificial
  Corresponds to a portion of human MEKK3
DGRRTFPRIR

(19) United States
(12) Patent Application Publication
(10) Pub. No.: US 2009/0156466 A1
(43) Pub. Date: Jun. 18, 2009

(54) Characterization of a MEKK binding site and uses thereof

(76) Inventor: Michael P. Scheid, Oakville (CA)

Correspondence Address: McCarthy Tetrault LLP, Box 48, Suite #4700 Toronto Dominion Bank Tower, TORONTO, ON, M5K 1E6, CA

(21) Appl. No.: 12/078,808

(22) Filed: Apr. 4, 2008

Related U.S. Application Data
(60) Provisional application No. 60/907,511, filed on Apr. 5, 2007.

(30) Foreign Application Priority Data
Apr. 5, 2007 (CA) 2,583,999

(51) Int. Cl. A61K 38/02; C12N 9/12

(52) U.S. Cl. 514/7; 435/194

(57) ABSTRACT

The present invention provides binding motifs, binding sites, peptides, and 14-3-3/MEKK complexes. Also provided are agents reactive with same, pharmaceutical compositions, molecules, and kits comprising same, and methods of producing same. The present invention further provides isolated nucleic acids and constructs comprising same, host cells comprising the constructs, and transgenic non-human animals. Additionally, the present invention provides methods of: (a) facilitating or inhibiting 14-3-3/MEKK interaction, (b) regulating MEKK activity, (c) identifying agents that interact with MEKK or 14-3-3, (d) detecting and monitoring the development of MEKK-mediated conditions, and (e) treating and/or preventing MEKK-mediated conditions.

### RELATED APPLICATIONS

This application claims the benefit of U.S. Provisional Application No. 60/907,511, filed Apr. 5, 2007, the contents of which are incorporated herein by reference. This application also claims the benefit of Canadian Application No. 2,583,999, filed Apr. 5, 2007, the contents of which are incorporated herein by reference.

### BACKGROUND OF THE INVENTION

Protein kinases are key regulators of extracellular cues to illicit gene expression. One group of protein kinases that is involved in a wide array of cellular functions is the mitogen-activated protein kinase (MAPK) family. It includes the well-characterized extracellular regulated kinase (ERK) 1/2, c-Jun N-terminal kinase (INK, also called SAPK), p38 MAPK, and the Big MAPK (BMK)/ERK5. These family members are all activated by hierarchical phosphorylation cascades involving upstream protein kinases called MAPKK and MAP(3)K. Numerous MAP(3)Ks have been identified, including MEKK1, MEKK2, MEKK3, MEKK4, tumor progression locus 2 (Tpl2), and transforming-growth-factor-B-activated kinase 1 (TAK1). The physiological roles of these signaling pathways depend upon the cellular context and history, and are regulated by a diverse set of signals, including stress, growth factors, and cell contact.

Of the MAP(3)K superfamily, MEKK2 and MEKK3 belong to the Ste11 family of MAP(3)K that mediate extracellular signals to numerous MAPK and NF-KB cascades (1-8). Phosphorylation has proved to be important in the regulation of MAP(3)Ks, and recent evidence suggests that both MEKK2 and MEKK3 undergo stimulus-specific phosphorylation on their activation loops (9,10). Phosphorylation at S519 and S526 increases MEKK2 and MEKK3 activity, respectively, towards downstream MAPKKs, and thus represents a switch to control MEKK2/3 pathway activation. In addition to S526, MEKK3 is phosphorylated at S166 and S337 (11,12). These residues fall within the canonical AGC phosphorylation motif of R-X-R-X-X-S, which suggests that they may be targets for AGC kinases, including Akt/PKB, SGK, S6K, PKA, and RSK. Indeed, Chun and coworkers have shown that S337 can undergo phosphorylation in vitro by SGK, suggesting that SGK could be a physiological kinase of MEKK3 (12).

The 14-3-3 proteins are recognized as important cellular regulators that facilitate the interaction between phosphorylated proteins (13-15). The interaction between another MAP(3)K, Raf-1, and 14-3-3 exemplifies this complex regulation, with both activating and inactivating functions. For example, Raf-1 is inactivated by phosphorylation at S259, which promotes the association with 14-3-3 proteins and stabilizes Raf-1 in an inactive conformation that cannot be recruited and activated by Ras (16-18). In contrast, S621 phosphorylation mediates distinct 14-3-3 binding, which stabilizes Raf-1 in an active conformation (19-21).

MEKK3 has been shown to associate with 14-3-3 (10,22). However, prior to the present invention, the site of interaction was not known.

### SUMMARY OF THE INVENTION

In seeking to understand the regulation of MEKK3, the cross-talk that occurs with AGC kinases, and the interaction between MEKK3 and 14-3-3, the inventor has identified a novel MEKK3 phosphorylation site, 1294. Furthermore, the inventor has shown this site to be the residue that interacts with 14-3-3 proteins.

Accordingly, in one aspect, the present invention provides a binding motif of a MEKK polypeptide capable of binding a 14-3-3 polypeptide, in which the motif includes an amino acid, sequence that includes a threonine capable of phosphorylation. In one embodiment, the threonine is phosphorylated. By way of example, the 14-3-3 polypeptide may be selected from the following group of 14-3-3 isoforms expressed in mammals: gamma, epsilon, beta, zeta, sigma, theta, and tau. In another embodiment, the MEKK polypeptide is MEKK3 (e.g., human MEKK3) or MEKK2 (e.g., human MEKK2). In yet another embodiment, the amino acid sequence includes the sequence RRTFP. For example, the amino acid sequence may include the sequence GRRTFPR or the sequence YSDGRRTFPRIRR. In such embodiments, the phosphorylated threonine may correspond to a threonine residue at position 294 of human MEKK3 (isoform 2) or position 345 of human MEKK3 (isoform 1). In a further embodiment, the amino acid sequence may also include the sequence RKTFP. By way of example, the amino acid sequence may include the sequence GRKTFPR or the sequence YNDGRKTFPRARR. In such embodiments, the phosphorylated threonine may correspond to a threonine residue at position 283 of MEKK2.

In another aspect, the present invention provides an agent reactive with a binding motif of the invention. In one embodiment, the agent is an antibody. In another embodiment, the antibody is a monoclonal antibody. In still another embodiment, the agent is labeled with a detectable marker.

In a further aspect, the present invention provides a method of binding a 14-3-3 polypeptide to a MEKK polypeptide that includes a binding motif of the invention, including: (a) phosphorylating the threonine of the binding motif of the invention; and (b) contacting the phosphorylated binding motif with a 14-3-3 polypeptide.

In yet another aspect, the present invention provides a method of regulating activity of a MEKK polypeptide that includes the binding motif of the invention, including modulating phosphorylation of the threonine of the binding motif of the invention. In one embodiment, the phosphorylation is modulated in the presence of a 14-3-3 polypeptide.

In still another aspect, the present invention provides a method of regulating activity of a MEKK polypeptide that includes the binding motif of the invention, including contacting the MEKK polypeptide with an antagonist that binds the MEKK polypeptide at the binding motif of the invention. In one embodiment, the MEKK polypeptide is contacted with the antagonist in the presence of a 14-3-3 polypeptide. In another embodiment, the antagonist is an antibody, a small molecule, a peptide, or a peptide mimetic.

In a further aspect, the present invention provides a method of treating and/or preventing a MEKK-mediated condition in a subject, including modulating phosphorylation of a MEKK polypeptide in the subject, wherein the MEKK polypeptide includes the binding motif of the invention and phosphorylation of the threonine is modulated. In one embodiment, the phosphorylation is modulated in the presence of a 14-3-3 polypeptide.

In yet another aspect, the present invention provides a binding motif of MEKK3 capable of binding a 14-3-3 polypeptide, in which the binding motif includes an amino acid sequence including: (a) the sequence ²⁹²RRTFP²⁹⁶ of isoform 2 of the MEKK3, or an analogue or functional equivalent thereof, wherein T²⁹⁴ is capable of being phosphorylated; or (b) the sequence ³⁴³RRTFP³⁴⁷ of isoform 1 of the MEKK3, or an analogue or functional equivalent thereof, wherein T³⁴⁵ is capable of being phosphorylated. Also provided is a phosphorylated binding motif of MEKK3 capable of binding a 14-3-3 polypeptide, in which the binding motif includes an amino acid sequence including: (a) the sequence ²⁹²RRT*FP²⁹⁶ of isoform 2 of the MEKK3, or an analogue or functional equivalent thereof, where * denotes phosphorylation; or (b) the sequence ³⁴³RRT*FP³⁴⁷ of isoform 1 of the MEKK3, or an analogue or functional equivalent thereof, where * denotes phosphorylation.

In another aspect, the present invention provides a binding motif of MEKK2 capable of binding a 14-3-3 polypeptide, in which the binding motif includes an amino acid sequence including the sequence ²⁸¹RKTFP²⁸⁵ of the MEKK2, or an analogue or functional equivalent thereof, wherein T²⁸³ is capable of being phosphorylated. Also provided is a phosphorylated binding motif of MEKK2 capable of binding a 14-3-3 polypeptide, in which the binding motif includes an amino acid sequence including the sequence ²⁸¹RKT*FP²⁸⁵ of the MEKK2, or an analogue or functional equivalent thereof, where * denotes phosphorylation.

In still another aspect, the present invention provides a 14-3-3-polypeptide-binding site of MEKK3, including amino acid residue T294 or T345 of MEKK3. In one embodiment, the 14-3-3-polypeptide-binding site T294 or T345 is phosphorylated. In another embodiment, the 14-3-3-polypeptide-binding site includes amino acid residue T294, and further includes amino acid residues R292, R293, F295, and P296 of MEKK3. In yet another embodiment, the 14-3-3-polypeptide-binding site further includes amino acid residues R297, I298, R299, and R300 of MEKK3. In still another embodiment, the 14-3-3-polypeptide-binding site further includes amino acid residues Y288, S289, D290, and G291 of MEKK3.

In a further aspect, the present invention provides a 14-3-3-polypeptide-binding site of MEKK2, including amino acid residue T283 of MEKK2. In one embodiment, the 14-3-3-polypeptide-binding site T283 is phosphorylated. In another embodiment, the 14-3-3-polypeptide-binding site further includes amino acid residues R281, K282, F284, and P285 of MEKK2. In still another embodiment, the 14-3-3-polypeptide-binding site further includes amino acid residues R286, A287, R288, and R289 of MEKK2. In yet another embodiment, the 14-3-3-polypeptide-binding site further includes amino acid residues Y277, N278, D279, and G280 of MEKK2.

In another aspect, the present invention provides a molecule having a 14-3-3-polypeptide-binding site, in which the molecule includes at least 13 amino acid residues and the 14-3-3-polypeptide-binding site includes the contiguous polypeptide sequence YSDGRRTFPRIRR. In one embodiment, the molecule T is phosphorylated. In another embodiment, the molecule is a portion of MEKK3. Also provided is a molecule having a 14-3-3-polypeptide-binding site, in which the molecule includes at least 13 amino acid residues and the 14-3-3-polypeptide-binding site includes the contiguous polypeptide sequence YNDGRKTFPRARR. In one embodiment, the molecule T is phosphorylated. In another embodiment, the molecule is a portion of MEKK2.

In yet another aspect, the present invention provides a method of identifying an agent which interacts with MEKK3, including: (a) contacting MEKK3 with a candidate agent; and (b) assessing the ability of the candidate agent to bind to MEKK3 at a binding site including amino acid residues R292, R293, T294, F295, and P296 or R343, R344, T345, F346, and P347 of MEKK3. In one embodiment, T294 or T345 is phosphorylated. Also provided is an agent identified by this method.

In still another aspect, the present invention provides a method of identifying an agent which interacts with MEKK2, including: (a) contacting MEKK2 with a candidate agent; and (b) assessing the ability of the candidate agent to bind to MEKK2 at a binding site including amino acid residues R281, K282, T283, F284, and P285 of MEKK2. In one embodiment, T283 is phosphorylated. Also provided is an agent identified by this method.

In a further aspect, the present invention provides a peptide that modulates activity of a MEKK polypeptide, including an amino acid sequence that includes an amino acid capable of phosphorylation, wherein the amino acid capable of phosphorylation is threonine or serine (T/S). In one embodiment, the peptide is an amino acid capable of phosphorylation is threonine. In another embodiment, the peptide is a threonine/serine is phosphorylated. In yet another embodiment, the peptide MEKK polypeptide is MEKK3 (e.g., human MEKK3) or MEKK2 (e.g., human MEKK2). In a further embodiment, the peptide is an amino acid sequence including the sequence RXT/SZP, where X is any amino acid and Z is any hydrophobic amino acid. In a still further embodiment, the peptide is an amino acid sequence including the sequence RXTZP, the sequence RRTFP, the sequence GRRTFPRI, and/or the sequence YSDGRRTFPRIRR. In still another embodiment, the peptide is an amino acid sequence including the sequence RKTFP, the sequence GRKTFPRA, and/or the sequence YNDGRKTFPRARR. In another embodiment, the peptide inhibits interaction between the MEKK polypeptide and a 14-3-3 polypeptide (e.g., the gamma, epsilon, beta, zeta, sigma, theta, or tau isoforms of 14-3-3 polypeptide). In a further embodiment, the peptide inactivates the MEKK polypeptide. Also provided is a pharmaceutical composition including the peptide of the invention and a pharmaceutically-acceptable carrier, diluent, or excipient.

In another aspect, the present invention provides an agent reactive with the peptide of the invention. In one embodiment, the agent is labeled with a detectable marker. In another embodiment, the agent is an antibody (e.g., a monoclonal antibody). In a further embodiment, the agent is an antibody that specifically binds to the peptide. Also provided is a method of producing a antibody of the invention, including: (a) immunizing a mammal with a peptide of the invention; and (b) purifying antibody from a tissue of the mammal or from a hybridoma made using tissue of the mammal. An isolated nucleic acid molecule encoding the peptide of the invention is also provided, a long with a recombinant nucleic acid construct including the nucleic acid molecule of the invention operably linked to an expression vector and a host cell including the recombinant nucleic acid construct of the invention. In addition, the present invention provides a method of producing the peptide of the invention, including: (a) culturing the at least one host cell of the invention, under conditions allowing expression of the peptide; and (b) recovering the peptide from the at least one host cell or culture medium thereof.

In still another aspect, the present invention provides a transgenic non-human animal whose genome includes a disruption in its endogenous MEKK2 or MEKK3 gene, wherein the disruption occurs within the nucleic acid sequence that encodes ²⁷⁷YNDGRKTFPRARR²⁸⁹ of MEKK2 or ²⁸⁸YSDGRRTFPRIRR³⁰⁰ or ²³⁹YSDGRRTFPRIRR³⁵¹ of MEKK3, respectively. In one embodiment, the transgenic animal is a knock-in mutation disruption of an alanine at position 283 of MEKK2 or position 294 or 345 of MEKK3.

In a further aspect, the present invention provides a peptide that modulates activity of a MEKK polypeptide in a cell, including about 4 to about 30 amino acids containing a sequence derived from a MEKK3 sequence surrounding T294 or T345, wherein the amino acid residue in the peptide that corresponds to T294 or T345, respectively, is capable of phosphorylation, or an analogue or functional equivalent thereof. Also provided is a peptide that modulates activity of a MEKK polypeptide in a cell, including about 4 to about 30 amino acids containing a sequence derived from a MEKK2 sequence surrounding T283, wherein the amino acid residue in the peptide that corresponds to T283 is capable of phosphorylation, or an analogue or functional equivalent thereof.

In yet another aspect, the present invention provides a method of treating and/or preventing a MEKK-mediated condition in a subject, including administering to the subject a peptide including amino acid sequence RXT/SZP, where X is any amino acid, Z is any hydrophobic amino acid, and T/S is capable of phosphorylation. In one embodiment, the MEKK is MEKK3 (e.g., human MEKK3) or MEKK2 (e.g., human MEKK2). In another embodiment, the subject is a human. In still another embodiment, the T/S is phosphorylated. In a further embodiment, the peptide modulates MEKK activity in the subject, thereby treating and/or preventing the MEKK-mediated condition in the subject. In yet another embodiment, the peptide inactivates a MEKK polypeptide in the subject, thereby treating and/or preventing the MEKK-mediated condition in the subject. By way of example, the peptide may inhibit interaction between a 14-3-3 polypeptide and a MEKK polypeptide in the subject, thereby treating and/or preventing the MEKK-mediated condition in the subject. Exemplary MEKK-mediated conditions include, without limitation, chronic inflammatory disease and a condition associated with expression of pro-inflammatory cytokines. In one embodiment, the MEKK-mediated condition is an autoimmune disorder (e.g., arthritis, asthma, or lupus). The peptide may be administered orally, parenterally, transdermally, intranasally, by pulmonary administration, or by osmotic pump.

In still another aspect, the present invention provides a method of determining whether a subject has a MEKK-mediated condition or is at increased risk of developing a MEKK-mediated condition, including assaying a diagnostic sample of the subject for a MEKK3 polypeptide in which amino acid residue T294 or T345 is phosphorylated or for a MEKK2 polypeptide in which amino acid residue T283 is phosphorylated, wherein detection of the presence of the phosphorylated amino acid residue is indicative that the subject has a MEKK-mediated condition or is at increased risk of developing a MEKK-mediated condition. In one embodiment, the diagnostic sample is assayed using an agent reactive with phosphorylated amino acid residue T294 or T345 in MEKK3 or phosphorylated amino acid residue T283 in MEKK2. In another embodiment, the agent is labeled with a detectable marker. In still another embodiment, the agent is an antibody. In a further embodiment, the antibody is labeled with a detectable marker.

In a further aspect, the present invention provides a method of predicting whether a subject would be responsive to treatment for a MEKK-mediated condition, wherein the treatment includes administering to the subject a peptide including amino acid sequence RXT/SZP, where X is any amino acid, T/S is phosphorylated, and Z is any hydrophobic amino acid, the method including assaying a diagnostic sample of the subject for a MEKK3 polypeptide in which amino acid residue 294 or 345 is phosphorylated or for a MEKK2 polypeptide in which amino acid residue 283 is phosphorylated, wherein the presence of the phosphorylated amino acid residue is indicative that the subject would be responsive to the treatment.

In yet another aspect, the present invention provides a kit for determining whether a subject has a MEKK-mediated condition or is at increased risk of developing a MEKK-mediated condition, including: (a) a reagent that detects the presence of a MEKK3 polypeptide in which amino acid residue T294 or T345 is phosphorylated and/or a MEKK2 polypeptide in which amino acid residue T283 is phosphorylated; and (b) instructions for using the kit to determine whether the subject has a MEKK-mediated condition or is at increased risk of developing a MEKK-mediated condition. In one embodiment, the kit further includes a container.

In still another aspect, the present invention provides a method of facilitating interaction between a 14-3-3 polypeptide and MEKK3, including phosphorylating amino acid residue T294 or T345 on MEKK3. Also provided is a method of facilitating interaction between a 14-3-3 polypeptide and MEKK2, including phosphorylating amino acid residue T283 on MEKK2.

In addition, the present invention provides, in another aspect, a method of inhibiting interaction between a 14-3-3 polypeptide and MEKK3, which includes dephosphorylating amino acid residue T294 or T345 on MEKK3. Also provided is a method of inhibiting interaction between a 14-3-3 polypeptide and MEKK2, which includes dephosphorylating amino acid residue T283 on MEKK2.

In a further aspect, the present invention provides a method of inhibiting interaction between a 14-3-3 polypeptide and a MEKK polypeptide in cells of a subject, including contacting the cells with an amount of a phosphopeptide effective to inhibit the interaction, wherein the phosphopeptide includes the amino acid sequence RXT/SZP, where X is any amino acid, T or S is phosphorylated, and Z is any hydrophobic amino acid. In one embodiment, the MEKK polypeptide is MEKK3 (e.g., human MEKK3) or MEKK2 (e.g., human MEKK2). In another embodiment, the subject is a human. In a further embodiment, the amount of phosphopeptide effective to inhibit interaction between the 14-3-3 polypeptide and the MEKK polypeptide is an amount of phosphopeptide effective to treat or prevent a MEKK-mediated condition in the subject. In still another embodiment, an amino acid residue 294 or 345 of MEKK3 is phosphorylated. Also provided is a composition including a 14-3-3 polypeptide and MEKK2, wherein amino acid residue 283 of MEKK2 is phosphorylated.

In another aspect, the present invention provides a method of identifying an agent which interacts with a 14-3-3-polypeptide, including: (a) contacting a candidate agent with the 14-3-3 polypeptide, in the presence of MEKK3 having a phosphorylated T294 or T345 amino acid residue; and (b) assessing the ability of the candidate agent to inhibit a 14-3-3-polypeptide-MEKK3 interaction. Also provided is an agent identified by this method.

In yet another aspect, the present invention provides a method of identifying an agent which interacts with a 14-3-3-polypeptide, including: (a) contacting a candidate agent with the 14-3-3 polypeptide, in the presence of MEKK2 having a phosphorylated T283 amino acid residue; and (b) assessing the ability of the candidate agent to inhibit a 14-3-3-polypeptide-MEKK2 interaction. Also provided is an agent identified by this method

Additional aspects of the present invention will be apparent in view of the description which follows.

### BRIEF DESCRIPTION OF THE FIGURES

The invention will now be described in relation to the drawings, the contents of which are discussed below.

FIG. 1 depicts molecular-weight bandshift of MEKK3 and mutants on 118:1 acrylamide:bis-acrylamide SDS-PAGE. HEK 293 cells were transfected with FLAG-MEKK3 or MEKK3 with the indicated point mutations. Cells were lysed, and proteins were resolved by SDS-PAGE using a high-ratio 118:1 acrylamide:bis-acrylamide SDS-PAGE gel to separate phosphorylated and non-phosphorylated species. Proteins were transferred to PVDF, and detected by anti-FLAG immunoblotting.

FIG. 2 shows that immunoprecipitation of 14-3-3 requires MEKK3 catalytic activity. A, B. HEK 293 cells were transfected with wildtype (wt) MEKK3, K391M MEKK3, or S526A MEKK (shown in B) or empty vector. After 24 hours, cells were solubilized, and FLAG-MEKK3 was immunoprecipitated with anti-FLAG M2-conjugated agarose. Proteins were resolved by SDS-PAGE, and immunoblotted with anti-FLAG antibody or anti-14-3-3β antibody (which recognizes the family of 14-3-3 proteins). C. HEK 293 cells were transfected with wt MEKK3 for 24 hours, and then treated with staurosporine (1 μM, 5 μM, or 10 μM) for 20 min prior to solubilization. MEKK3 and 14-3-3 were detected as in A.

FIG. 3 shows that MEKK3-14-3-3 interaction does not require S166, S250, S337, or S357. HEK 293 cells were transfected with wt MEKK3, K391M MEKK3, TM (S166/S250/S337A compound mutation), S357A MEKK, or empty vector. After 24 hours, cells were solubilized, and FLAG-MEKK3 was immunoprecipitated with anti-FLAG M2-conjugated agarose. Proteins were resolved by SDS-PAGE, and immunoblotted with anti-FLAG antibody or anti-14-3-3β antibody.

FIG. 4 illustrates the identification of T294 as a site of phosphorylation. A. WT MEKK3 or K391 MEKK3 was expressed in HEK 293 cells for 24 hours, and then transferred for 4 hours to phosphate-free medium containing 1 mCi/ml ³²P-orthophosphate. FLAG-MEKK3 was immunoprecipitated with anti-FLAG M2-conjugated agarose, fractionated by SDS-PAGE, and visualized by autoradiography. B. ³²P-labeled MEKK3 was hydrolyzed with HCl, and resolved by two-dimensional electrophoresis. ³²P-labeled amino acids were visualized by autoradiography; co-migrating cold amino acids were visualized by ninhydrin staining. C. ³²P-labeled wt MEKK3 and K391M MEKK3 were digested with trypsin, and resolved by two-dimensional electrophoresis/chromatography as described in the Examples. The synthetic phosphopeptide pTFPR (1 μg) was included in the wt MEKK3 sample, and visualized by ninhydrin staining (indicated by the circle at spot A). D. Pre-immune serum, or serum from rabbits immunized for 56 days with the synthetic phosphopeptide YNDGRR(pT)FPRIRR coupled to KLH, was used to immunoblot lysates from cells expressing empty vector, wt MEKK3, K391M MEKK3, or T294A MEKK3. Total FLAG-MEKK3 was visualized using anti-FLAG antibody.

FIG. 5 shows that phosphorylated T294 mediates 14-3-3 binding. A. HEK 293 cells were transfected with wt MEKK3, T294A MEKK, or empty vector. The next day, cells were solubilized, and FLAG-MEKK3 was immunoprecipitated with anti-FLAG M2-conjugated agarose. Proteins were resolved by SDS-PAGE, and immunoblotted using anti-FLAG (upper panel) or anti-14-3-3, (lower panel) antibody. B. HEK 293 cells were transfected with wt MEKK3 for 24 hours. Anti-FLAG M2-conjugated agarose was added to the cell lysates, along with 200 μM peptide 1 (DGRRTFPRIR) or phosphopeptide 1 (DGRR(pT)FPRIR), or 200 μM peptide 2 (YNDGRRTFPRIRR) or phosphopeptide 2 (YNDGRR(pT)FPRIRR), as indicated. After overnight mixing, the agarose beads were pelleted, and bound proteins were resolved by SDS-PAGE and then immunoblotted with anti-FLAG antibody (upper panel) or anti-14-3-30 antibody (lower panel).

FIG. 6 illustrates MAPK signaling by T294A MEKK3. HEK 293 cells were transfected with HA-ERK (100 ng) and wt MEKK3, K391M MEKK3, or T294A MEKK. 24 hours later, cells were solubilized, resolved by SDS-PAGE, and immunoblotted with: A. anti-HA, anti-phospho-ERK, and anti-FLAG; and B. anti-phospho-p38 MAPK and anti-phospho-MKK4.

FIG. 7 depicts sequence alignment between human MEKK2 and MEKK3 surrounding T283/T294.

### DETAILED DESCRIPTION OF THE INVENTION

MEKK2 and MEKK3 play important roles in the activation of numerous MAPK and NF-κB signaling pathways following cellular stress and activation by pro-inflammatory cytokines. For example, MEKK3 is essential for inflammatory gene expression that is induced downstream of TNF receptor-1, IL-1 receptor, and Toll-like receptor activation through the activation of JNK, p38 MAPK, and NF-KB (26,28). Accordingly, the mechanisms that control MEKK2 and MEKK3 activation, and their target specificities, are subjects of intense interest.

Specificity of signaling is coordinated through hierarchical phosphorylation cascades that are regulated, in part, through association between signaling proteins. The well-known phosphoprotein-binding molecule, 14-3-3, plays many roles in controlling the specificity, signal strength, and spatial localization of numerous pathway components (29). 14-3-3 has been shown to interact with MEKK3, but the site of interaction, or the biological significance, has not yet been determined.

In the present investigation, the inventor performed tryptic mapping, phosphoamino acid analysis, and immunoblot analysis with a phosphospecific antibody to identify T294 as a novel site of MEKK3 phosphorylation. Mutation of this residue to alanine abolished 14-3-3 interaction, as did incubation of MEKK3 with a synthetic peptide containing phosphorylated, but not the unphosphorylated, T294 peptide. These novel and surprising results demonstrate that T294 is the primary MEKK3 site of 14-3-3 interaction.

As disclosed herein, the inventor determined that phosphorylation of T294 was dependent upon the catalytic activity of MEKK3, since mutation of the catalytic lysine at position 391 significantly diminished phosphorylation of T294 and dramatically reduced the interaction between MEKK3 and 14-3-3. This suggests that phosphorylation of T294 occurs by autophosphorylation. However, it is also possible that a downstream kinase, activated by MEKK3, phosphorylates T294. Phosphorylation of the activation loop S526 was also necessary for 14-3-3 binding, but it is unlikely that 14-3-3 directly interacts with this site. S526 phosphorylation was necessary for kinase activity, and mutation of this residue functionally mimicked kinase-dead mutation, K391M.

The role that 14-3-3 association with MEKK3 plays is of considerable interest. With Raf-1, 14-3-3 association to S259 and S621 plays opposing roles in regulating Raf-1 activity (30). To probe the role of 14-3-3 association with MEKK3, the inventor examined the ability of MEKK3 to induce the phosphorylation of the MAPKs, ERK and p38 MAPK, and the MAP(2)K, MKK4, in cells expressing wildtype and T294A MEKK3. In these experiments, phosphorylation of ERK, p38 MAPK, and MKK4 appeared to be similar between wt and T294A MEKK3, indicating that T294A MEKK3 retains catalytic activity in the absence of 14-3-3 binding.

These results suggest that, unlike the activation loop S526A mutation of MEKK3, which abolishes kinase activity and results in a total loss of ERK phosphorylation (9,10; and data not shown), 14-3-3 binding to phospho-T294 might play a more subtle role in regulating downstream pathway activation. Indeed, it has been suggested that 14-3-3 interaction with MEKK3 could serve to protect S526 phosphorylation, thereby preserving kinase activity (10). Additional work has also shown that dimerization of MEKK2 and MEKK3 is required for their full activation (31), and it is possible that 14-3-3 binding could play a role in this process. Thus, endogenous MEKK3, which undergoes stimulus-dependent S526 autophosphorylation, could require 14-3-3 interaction for maximal activity. Alternatively, 14-3-3 binding could play a role in MEKK3 pathway specificity by coupling MEKK3 to signal-dependent binding partners. Correlation of T294 phosphorylation of endogenous MEKK3 proteins with upstream signals will be possible using the phospho-T294 antibody that the inventor has generated in accordance with methods disclosed herein.

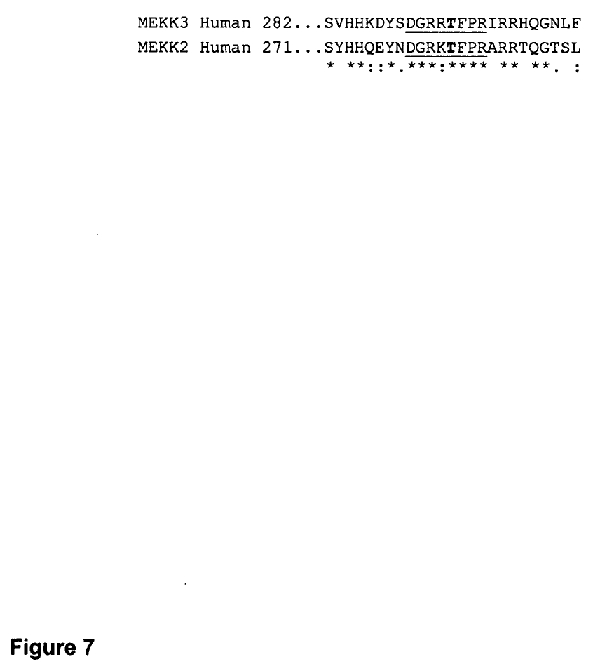

The closely-related MEKK2 protein kinase shares significant homology to MEKK3, but may have distinct signaling functions. MEKK2 is highly homologous to MEKK3 in the amino acid sequence surrounding the equivalent site of T283, suggesting that MEKK2 might associate with 14-3-3 at T283 (FIG. 7). Notably, this sequence is not present in any other protein kinase with any degree of homology. Therefore, the 14-3-3-binding motif at T283M94 is likely unique to the MEKK2/3 family.

The present invention is described in the following Examples, which are set forth to aid in the understanding of the invention, and should not be construed to limit in any way the scope of the invention as defined in the claims which follow thereafter.

### EXAMPLES

### Example 1

### Cell Cultures

HEK 293 cells were obtained from the American Type Culture Collection, and maintained in Dulbecco's modified Eagles medium (DMEM) supplemented with 10% fetal bovine serum and antibiotics at 37° C., 5% CO₂, and humidity.

### Example 2

### Plasmids and Mutogenesis

Human MEKK3 cDNA was cloned from I.M.A.G.E. clone id # 7939519 by PCR, and inserted into pCMV10-3×FLAG to introduce an N-terminal FLAG epitope. Mutagenesis of pCMV10-3×FLAG-MEKK3 was performed using a Quickchange kit (Strategene), and mutations were sequence verified.

### Example 3

### cDNA Transfection

HEK 293 cells were plated onto 35-mm-diameter dishes at 80% confluency, and transfected with 100 to 500 ng of cDNA using Lipofectamine-2000 (Gibco-BRL) following the manufacturer's protocol. Transfection medium was removed and replaced with complete DMEM overnight.

### Example 4

### Cell Lysis, Immunoprecipitation, and Immunoblotting

Cells were lysed in 50 mM Tris-HCl, pH 7.4, 1% Triton X-100, 25 mM NaF, 25 mM β-glycerophosphate, 5 mM EDTA, 0.05% SDS, 1.0 μg/ml microcystin LR, and protease inhibitors. Anti-FLAG M2 antibody, conjugated to agarose beads (Sigma), was added to lysates and incubated overnight at 4° C. Beads were washed three times with lysis buffer, and proteins were eluted with 200 μl of lithium dodecyl sulfate (LDS) sample buffer heated to 70° C. for 10 minutes. Portions of the lysates prior to immunoprecipitation were also boiled with LDS-containing sample buffer. Lysates and immunoprecipitations were fractionated by SDS polyacrylamide gel electrophoresis (PAGE). Proteins were transferred to a polyvinylidene difluoride (PVDF) membrane, blocked in 5% skim milk for 30 min, and probed with the appropriate antibody overnight at 4° C. Secondary decoration with horseradish-peroxidase-conjugated anti-rabbit or anti-mouse antibodies was performed at room temperature for 1.5 hours. Proteins were visualized using ECL, according to the manufacturer's protocol (Amersham). For blotting using the Licor Odyssey infrared imager, membranes were probed with IR680-anti-rabbit and IR800-anti-mouse secondary antibodies. FLAG was stained with anti-FLAG M2 mouse monoclonal (Sigma), and 14-3-3 isoforms were detected with anti-14-3-3β (H8; Santa Cruz Biotechnology).

### Example 5

### Metabolic Labeling

HEK 293 cells were plated onto 35-mm-diameter dishes at 80% confluency, and transfected with 500 ng of pCMVI0-FLAG-MEKK3 using Lipofectamine-2000 (Gibco-BRL) according to the manufacturer's protocol. After 24 hours, cells were placed in phosphate-free DMEM medium with 1 mCi/ml ³²P-labeled orthophosphate at 37° C. for 4 h. FLAG-MEKK3 was immunoprecipitated from detergent-solubilized lysates, and fractionated on an 8% gel. ³²P-labeled MEKK3 was detected by autoradiography.

### Example 6

### Tryptic Digestion, Two-Dimensional Phosphopeptide Mapping, and Phosphoamino Acid Analysis

³²P-metabolically-labeled FLAG-MEKK3 was isolated as described above, excised from the gel, and digested with 10 μg/ml tosylphenylalanylk chloromethyl ketone-treated trypsin (Promega) in 50 mM (NH₄)HCO₃, pH 7.8, overnight at 37° C. Gel fragments were pelleted by centrifugation, and the remaining supernatant was transferred to clean tubes and dried under vacuum. Dried peptides were resuspended in 50 μL performic acid, incubated on ice for 2 hours, and dried under vacuum. Peptides were washed with diminishing volumes of water, and resuspended in 5 μl of pH 1.9 electrophoresis buffer. Electrophoresis was performed on 200-μm microcrystalline cellulose plates (Kodak) at 1000 V, 7° C., for 30 min. The plates were chromatographed in the second dimension in chromatography buffer (n-butanol/pyridine/acetic acid/water, 32.5:25:5:20). Plates were dried, and phosphopeptides were visualized using an FX-Imager (BioRad) or film autoradiography. If cold synthetic phosphopeptides were also run, these were visualized with ninhydrin staining. Phosphoamino acid analysis was performed by hydrolyzing ³²P-labelled MEKK3 in 500 μl of 6 N HCl heated to 110° C. for 60 min. The HCl was removed under vacuum, and the phosphoamino acids were washed with diminishing volumes of water. Separation was performed on cellulose plates using buffer consisting of 0.5% pyridine and 5% acetic acid at 1500 V, 7° C., for 20 min in one direction, and then the plates were allowed to dry. Once dry, phosphoamino acids were separated in a second dimension using pH 3.5 buffer at 1600V, 7° C., for 13 min. ³²P-labeled phosphoamino acids were detected by autoradiography. In each of the samples, 1 μg of a mixture of phospho-L-serine, phospho-L-threonine, and phospho-L-tyrosine was also added; this was visualized by ninhydrin staining.

### Example 7

### Peptide Competition Assay

Peptide 1 (DGRRTFPRIR) and phosphopeptide 1 (DGRR(pT)FPRIR) corresponding to the sequence surrounding T294 were synthesized by Genscrip Corp (New Jersey). Peptide 2 (YNDGRRTFPRIRR) and phosphopeptide 2 (YNDGRR(pT)FPRIRR) were generated by Open Biosystems (Huntsville, Ala.). Lysates containing FLAG-MEKK3 were incubated with 200 μM of peptide or phosphopeptide where indicated. Anti-FLAG M2 agarose was added and the mixtures were incubated overnight at 4° C. Beads were washed three times and proteins were eluted and resolved by SDS PAGE. Co-immunoprecipitated 14-3-3 was detected using a pan-specific anti-14-3-3 antibody (Santa Cruz).

Discussed below are results obtained by the inventor in connection with the experiments of Examples 1-7:

### MEKK3 is Phosphorylated on Multiple Residues in Cells.

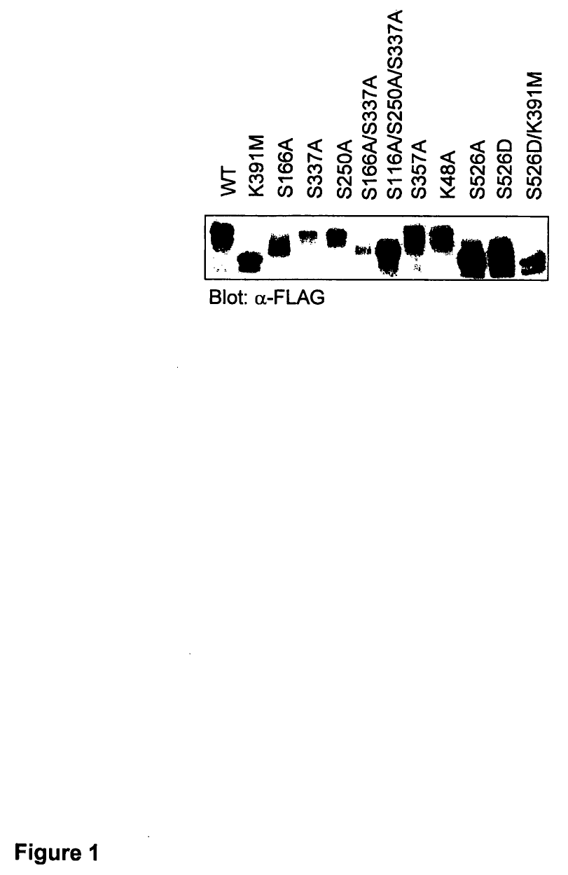

MEKK3 is a phosphoprotein (9-12), but the extent and physiological significance of phosphorylation of MEKK3 is not fully understood. In an initial survey, the inventor expressed wildtype (wt) MEKK3 and MEKK3 with various mutations in HEK293 cells, and resolved detergent-solubilized lysates on a high-ratio acrylamide:bis-acrylamide gel. MEKK3 resolved as numerous species over a 10-kDa range, with the majority of the wildtype protein migrating as a single band (FIG. 1). The mobility of MEKK3 was dramatically increased with introduction of a kinase-dead mutation, K391M, suggesting that MEKK3 autophosphorylates on a number of residues in cells. MEKKK3 expressed in cells treated with staurosporine, a broadly-specific serine/threonine kinase inhibitor, also reduced the MEKK3 band shift (data not shown), suggesting that staurosporine inhibits MEKK3 or one or more kinases upstream of MEKK3. Treatment with a PI3K inhibitor, wortmannin, had no effect on mobility shift, nor did the MEK1/2 inhibitor, U0126 (data not shown).

Next, the three known sites of phosphorylation—S166, S337, and S526—were individually mutated to alanine, and each allele was expressed in cells. The mobility shift of S166A was reduced compared with wildtype MEKK3 (FIG. 1), confirming that this site is likely phosphorylated in cells, as previously suggested (11). The S337A mutation did not affect mobility shift on SDS-PAGE. Finally, S526A reduced the mobility shift of MEKK3, comparable to K391M (FIG. 1). The loss of mobility shift of S526A was partially restored with the introduction of a phosphomimetic glutamic acid, S526D. The rescued mobility shift of S526D was completely abolished upon compounding this mutation with the kinase-dead lysine mutation, MEKK3 S526D/K391M (FIG. 1). This suggested that S526D was permissive for autophosphorylation on additional, distinct sites.

The inventor also introduced mutations at several other potential sites of phosphorylation, based on sequence similarity to known kinase phosphorylation motifs. These included S236, S250, and S357 (Table 1). None of these point mutations altered the mobility shift of MEKK3 (FIG. 1). Thus, the difference in mobility shift between kinase-dead MEKK3 and wildtype MEKK3, as well as the observation that S526D induced a mobility shift to a greater degree than S526D/K391M, suggested that MEKK3 autophosphorylates on one or more sites.

TABLE 1
Verified (bold) and potential (italicized) MEKK3 phosphorylation
sites and comparison with Mode 1 14-3-3 binding consensus motif.

| Site | Sequence |
|---|---|
| Mode 1 | |
| Consensus | XXRXXSXPX |
| Binding Site | |
| S166 | RSRHLSVSS |
| S250 | MSRARSFPD |
| S236 | LDRSADSPS |
| S337 | RGRLRSADS |
| S356 | PTKSPSAPI |
| T294 (this | NDGRRTFPR |
| study) | |

### Autophosphorylation Mediates 14-3-3-MEKK3 Association.

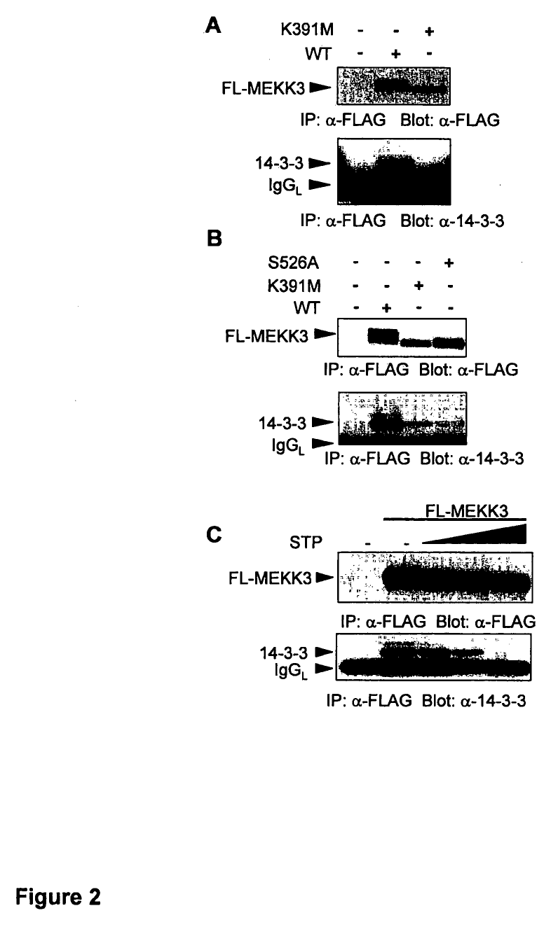

The phosphoprotein binding molecule 14-3-3 is involved in regulating a diverse set of cellular proteins by altering activity, location, and stabilization (23,24). MEKK3 has been shown to associate with 14-3-3 isoforms (22), but the mechanism and significance of this interaction is unknown. The inventor considered the possibility that MEKK3 autophosphorylation might play a role in mediating MEKK3-14-3-3 interaction, and found that MEKK3 co-immunoprecipitated with one or more endogenous 14-3-3 isoforms (FIG. 2a).

The family of 14-3-3 proteins was detected by immunoblotting with a pan-specific 14-3-3 antibody raised against a common sequence present in all family members. This interaction was specific for catalytically-active MEKK3, since the kinase-dead K391M mutant did not co-immunoprecipitate 14-3-3 (FIG. 2a). In addition, the intact S526 residue in the catalytic site, which is required for MEKK3 activity (9,10), was also essential for 14-3-3 association, since mutation to alanine prevented 14-3-3 co-immunoprecipitation (FIG. 2b). This suggested that MEKK3 activity was required for 14-3-3 association, possibly as a consequence of cis- or trans-autophosphorylation. The kinase inhibitor, staurosporine, also disrupted MEKK3-14-3-3 interaction (FIG. 2c), again indicating that kinase activity—either MEKK3 itself or an upstream kinase—was necessary for 14-3-3 association.

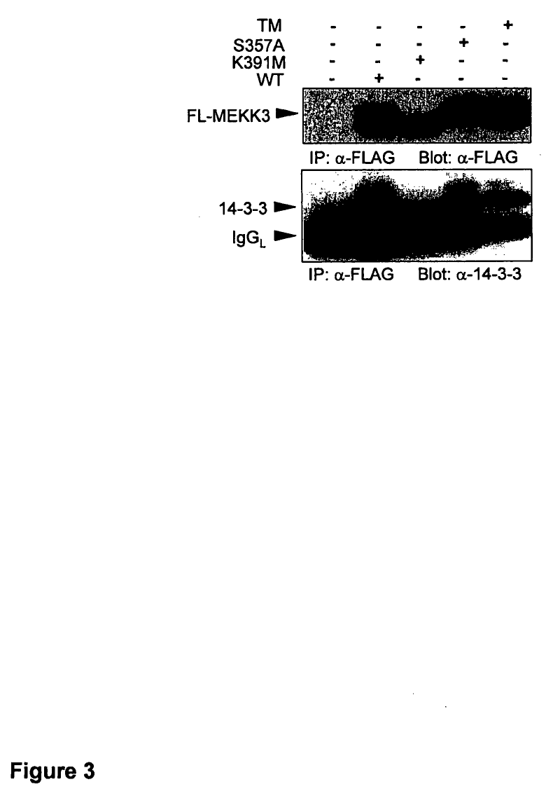

In addition to S526, two other sites of phosphorylation of MEKK3 have been identified: S166 and S337 (11). Another site, S250, lies between S166 and S337, and resembles a Mode 1 14-3-3 binding site. The inventor speculated that these three sites might act co-operatively in binding with 14-3-3, and that this might not be revealed when testing single point mutations. To test this possibility, the inventor created a triple mutation consisting of S166A/S250A/S337A. The MEKK3 triple mutation, in which S166, S250, and S337 were changed to alanine, co-immunoprecipitated 14-3-3 in a manner similar to that of wildtype MEKK3 (FIG. 3). This result conclusively demonstrated that phosphorylation of S166, S250, and S337 is not a requirement for regulating MEKK3-14-3-3 binding.

The inventor also tested other potential 14-3-3 binding sites, including S236 and S357. These sites could be Mode 1 14-3-3 binding sites based on sequence similarity to known 14-3-3 binding motifs (Table 1). However, mutation of S357 to alanine (FIG. 3) or S236 to alanine (not shown) did not prevent 14-3-3 co-immunoprecipitation. Taken together, the inventor's results indicate that 14-3-3 binding required phosphorylation of sites on MEKK3 distinct from S166, S236, S250, S337, and S357.

MEKK3 T294 is Identified as a Novel Site of Phosphorylation.

The inventor's observations of the mobility shift of MEKK3 and mutants, and the profile of 14-3-3 co-immunoprecipitation, suggested that MEKK3 could be phosphorylated on residues in addition to the confirmed sites of S166, S337, and S526. The inventor also predicted that some of these additional sites could be a result of autophosphorylation, and might mediate MEKK3-14-3-3 interaction.

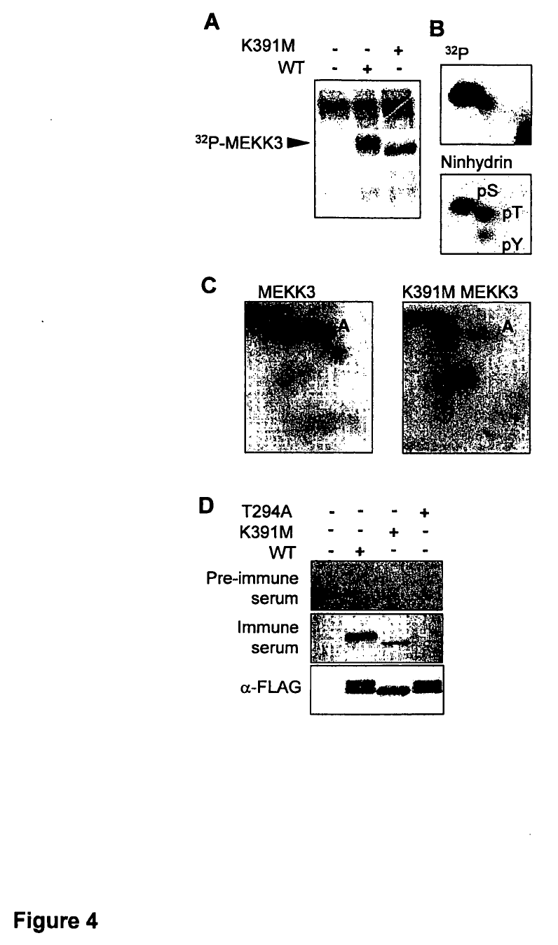

In order to identify the additional unknown site(s) of phosphorylation, the inventor expressed wt MEKK3 and K391M MEKK3, and labeled cells with ³²P-orthophosphate (FIG. 4a). Significant 32ₚ was incorporated into each of the expressed proteins, including K391M MEKK3, suggesting that both the active kinase and inactive kinase are targets of upstream kinases.

Next, the inventor performed phosphoamino acid analysis on the ³²P-labelled MEKK3 (FIG. 4b). This revealed a mixture of phosphoserine and phosphothreonine, indicating that MEKK3 was phosphorylated in cells on both amino acids. Tyrosine phosphorylation was not detected. Serine phosphorylation was expected; however, threonine phosphorylation was surprising.

The inventor examined the MEKK3 sequence to identify potential sites of threonine phosphorylation. The threonine residue at 294, within the sequence 291 . . . GRRT²⁹⁴FPRI . . . 298 (of MEKK3 isoform 2; T345 of the longer MEKK3 isoform 1), was considered as a potential site of phosphorylation because of the arginine at −2, the phenylalanine at +1, and the proline at +2. This motif resembles the canonical phosphorylation motif for R/K-directed, basophilic kinases, and also resembles the Mode 1 site for 14-3-3 protein binding.

To test if this site was phosphorylated in cells, the inventor digested ³²P-labeled MEKK3 with trypsin, and resolved the tryptic fragments by two-dimensional electrophoresis and cellulose chromatography. The inventor included in the sample the synthetic peptide [phospho-T]FPR, corresponding to the unique tryptic fragment generated from 291 . . . GRRT²⁹⁴FPRI . . . 298. Wildtype MEKK3 generated numerous ³²P-labelled tryptic peptides (FIG. 4c). The synthetic peptide [phospho-f]FPR co-migrated exactly with Spot A, revealing the identity of this tryptic spot as [phospho-T]FPR. The phosphorylation of this tryptic fragment, along with several other spots, was greatly diminished in tryptic maps generated from K391M MEKK3 (FIG. 4c). The other spots surrounding Spot A were likely partial digestion of the sequence flanking T294, DGRR(pT)FRIRR. Trypsin inefficiently cleaves between two arginine residues, and this would result in tryptic peptides of various lengths and charges. Consistent with this, the inventor noted that the 2D-tryptic maps generated from T294A MEKK3 showed that many of these additional spots were abolished, including Spot A (data not shown).

To further confirm that T294 was a site of phosphorylation, the inventor generated a phosphospecific antibody to phospho-T294. Serum from rabbits immunized with the synthetic peptide YNDGRR(pT)FPRIRR, corresponding to phospho-1294, detected wt MEKK3, but not T294A MEKK3 (FIG. 4d). In addition, K391M MEKK3 was only weakly detected, consistent with the reduction in phosphorylation at T294 observed by tryptic mapping. Together, these results demonstrate that T294 is a site of threonine phosphorylation of MEKK3.

### MEKK3 T294 is Essential for 14-3-3 Binding.

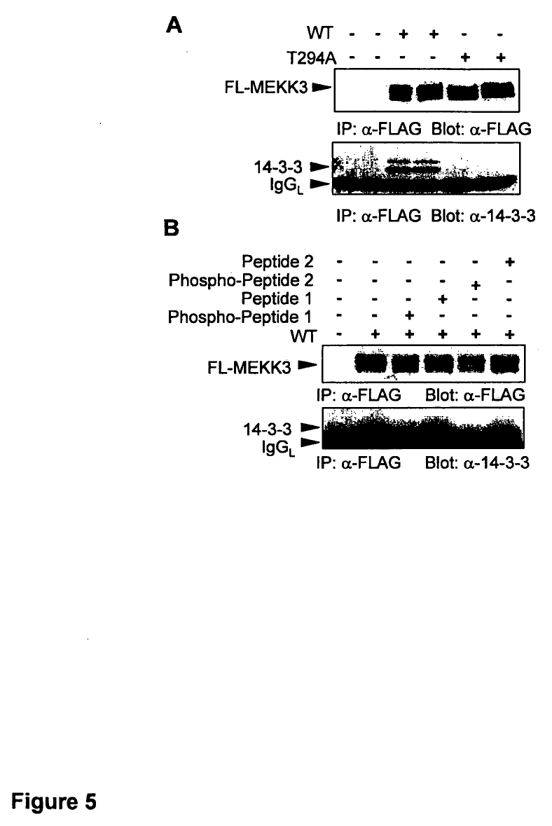

The inventor noted that the loss of threonine phosphorylation of K391M MEKK3 coincided with loss of 14-3-3 association. In addition, the inventor observed that the T294 residue resided within a potential Mode 1 14-3-3 binding site. Accordingly, the inventor tested the interaction of 14-3-3 with MEKK3 in which T294 was replaced with alanine. Mutation of T294 to alanine completely abolished 14-3-3 co-immunoprecipitation (FIG. 5a), suggesting that this site plays an important role in stabilizing 14-3-3—MEKK3 interaction. However, it remained possible that phosphorylation of T294 was acting as an enhancer to facilitate 14-3-3 binding to a distinct site.

To prove that T294 was the site of 14-3-3 binding, the inventor incubated wt MEKK3, prior to immunoprecipitation, with either phosphorylated or non-phosphorylated peptides corresponding to the sequence surrounding T294, and observed whether these peptides could compete with MEKK3 for 14-3-3 binding. The phosphorylated peptides abolished 14-3-3 co-immunoprecipitation, while the non-phosphorylated peptides had no effect on co-immunoprecipitation (FIG. 5b). Together, these experiments established that T294 was the primary site of 14-3-3 protein interaction.

### T294A MEKK3 and its Role in Activating Signaling Pathways.

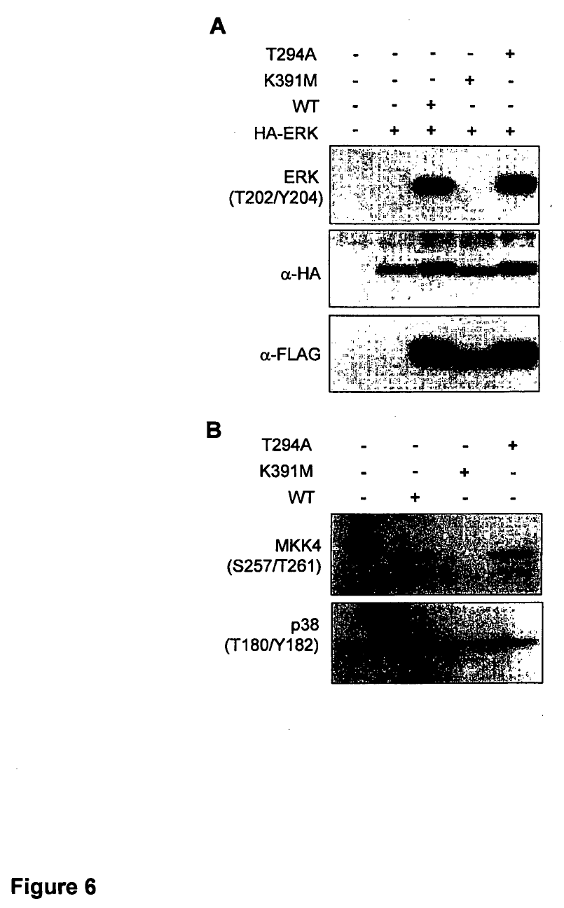

The inventor expressed MEKK3 in 293 cells, and examined the phosphorylation status of various MAPK and MAP(2)Ks. Previously, others established that MEKK3 is capable of activating components of the several MAPK pathways in cell lines and in primary cells (3, 6, 10, 25-27). In the inventor's experiments, both wildtype and T294A MEKK3, but not kinase-dead K391M MEKK3, induced the phosphorylation of ERK, MKK4, and p38 MAPK on activating sites within their catalytic domains (FIG. 6). These experiments demonstrate that T294A MEKK3 is both active and competent for MAPK pathway activation, and, unlike S621 of Raf-1, phosphorylation of T294 is not essential for MEKK3 activity.

While the foregoing invention has been described in some detail for purposes of clarity and understanding, it will be appreciated by one skilled in the art, from a reading of the disclosure, that various changes in form and detail can be made without departing from the true scope of the invention in the appended claims.

### REFERENCES

1. Deacon, K., and Blank, J. L. (1997) J Biol Chem 272(22), 14489-14496
2. Chao, T. H., Hayashi, M., Tapping, R. I., Kato, Y., and Lee, J. D. (1999) J Biol Chem 274(51), 36035-36038
3. Deacon, K., and Blank, J. L. (1999) J Biol Chem 274(23), 16604-16610
4. Zhao, Q., and Lee, F. S. (1999) J Biol Chem 274(13), 8355-8358
5. Uhlik, M. T., Abell, A. N., Johnson, N. L., Sun, W., Cuevas, B. D., Lobel-Rice, K. E., Home, E. A., Dell'Acqua, M. L., and Johnson, G. L. (2003) Nat Cell Biol 5(12), 1104-1110
6. Blank, J. L., Gerwins, P., Elliott, E. M., Sather, S., and Johnson, G. L. (1996) J. Biol. Chem. 271(10), 5361-5368
7. Sun, W., Kesavan, K., Schaefer, B. C., Garrington, T. P., Ware, M., Johnson, N. L., Gelfand, E. W., and Johnson, G. L. (2001) J. Biol. Chem. 276(7), 5093-5100
8. Ellinger-Ziegelbauer, H., Brown, K., Kelly, K., and Siebenlist, U. (1997) J. Biol. Chem. 272(5), 2668-2674
9. Zhang, D., Facchinetti, V., Wang, X., Huang, Q., Qin, J., and Su, B. (2006) Embo J 25(1), 97-107
10. Fritz, A., Brayer, K. J., McCormick, N., Adams, D. G., Wadzinski, B. E., and Vaillancourt, R. R. (2006) J Biol Chem 281(10), 6236-6245
11. Adams, D. G., Sachs, N. A., and Vaillancourt, R. R. (2002) Arch Biochem Biophys 407(1), 103-116
12. Chun, J., Kwon, T., Kim, D. J., Park, I., Chung, G., Lee, E. J., Hong, S. K., Chang, S. I., Kim, H. Y., and Kang, S. S. (2003) J Biochem (Tokyo) 133(1), 103-108
13. Muslin, A. J., Tanner, J. W., Allen, P. M., and Shaw, A. S. (1996) Cell 84(6), 889
14. Yaffe, M. B., Rittinger, K., Volinia, S., Caron, P. R., Aitken, A., Leffers, H., Gamblin, S. I., Smerdon, S. J., and Cantley, L. C. (1997) Cell 91(7), 961
15. Tzivion, G., Gupta, V. S., Kaplun, L., and Balan, V. (2006) Seminars in Cancer Biology 16(3), 203
16. Roy, S., McPherson, R. A., Apolloni, A., Yan, J., Lane, A., Clyde-Smith, J., and Hancock, J. F. (1998) Mol. Cell. Biol. 18(7), 3947-3955
17. Light, Y., Paterson, H., and Marais, R. (2002) Mol. Cell. Biol. 22(14), 4984-4996
18. Clark, G. J., Drugan, J. K., Rossman, K. L., Carpenter, J. W., Rogers-Graham, K., Fu, H., Der, C. J., and Campbell, S. L. (1997) J. Biol. Chem. 272(34), 20990-20993
19. Tzivion, G., Luo, Z., and Avruch, J. (1998) Nature 394(6688), 88
20. Thorson, J. A., Yu, L. W. K., Hsu, A. L., Shih, N.-Y., Graves, P. R., Tanner, J. W., Allen, P. M., Piwnica-Wormns, H., and Shaw, A. S. (1998) Mol. Cell. Biol. 18(9),
21. Ory, S., Zhou, M., Conrads, T. P., Veenstra, T. D., and Morrison, D. K. (2003) Current Biology 13(16), 1356
22. Fanger, G. R., Widmann, C., Porter, A. C., Sather, S., Johnson, G. L., and Vaillancourt, R. R. (1998) J Biol Chem 273(6), 3476-3483
23. Aitken, A. (2006) Seminars in Cancer Biology 16(3), 162
24. Yaffe, M. B. (2002) FEBS Letters 513(1), 53
25. Ellinger-Ziegelbauer, H., Brown, K., Kelly, K., and Siebenlist, U. (1997) J Biol Chem 272(5), 2668-2674
26. Huang, Q., Yang, J., Lin, Y., Walker, C., Cheng, J., Liu, Z.-g., and Su, B. (2004) Nat Immunol 5(1), 98
27. Chao, T.-H., Hayashi, M., Tapping, R. I., Kato, Y., and Lee, J.-D. (1999) J. Biol. Chem. 274(51), 36035-36038
28. Yang, J., Lin, Y., Guo, Z., Cheng, J., Huang, J., Deng, L., Liao, W., Chen, Z., Liu, Z.-g., and Su, B. (2001) Nat Immunol 2(7), 620
29. Jin, J., Smith, F. D., Stark, C., Wells, C. D., Fawcett, J. P., Kulkarni, S., Metalnikov, P., O'Donnell, P., Taylor, P., Taylor, L., Zougmnan, A., Woodgett, J. R., Langeberg, L. K., Scott, J. D., and Pawson, T. (2004) Current Biology 14(16), 1436
30. Wilker, E., and Yaffe, M. B. (2004) Journal of Molecular and Cellular Cardiology 37(3), 633
31. Cheng, J., Yu, L., Zhang, D., Huang, Q., Spencer, D., and Su, B. (2005) J. Biol. Chem. 280(14), 13477-13482

1. A binding motif of a MERK polypeptide capable of binding a 14-3-3 polypeptide, said motif comprising an amino acid sequence that includes a threo-nine capable of phosphorylation.

2. The binding motif of claim 1, wherein the threonine is phosphorylated.

3. (canceled)

4. The binding motif of claim 1, wherein the MEKK polypeptide is MEKK3.

5. The binding motif of claim 4, wherein the MEKK polypeptide is human MEKK3.

6. The binding motif of claim 4, wherein the amino acid sequence comprises the sequence RRTFP.

7. The binding motif of claim 6, wherein the amino acid sequence comprises the sequence GRRTFPR.

8. The binding motif of claim 7, wherein the amino acid sequence comprises the sequence YSDGRRTFPRIRR.

9. The binding motif of claim 4, wherein the threonine is phosphorylated.

10. The binding motif of claim 9, wherein the phosphorylated threonine corresponds to a threonine residue at position 294 of human MEKK3 (isoform 2) or position 345 of human MEKK3 (isoform 1).

11. The binding motif of claim 1, wherein the MEKK polypeptide is MEKK2.

12. The binding motif of claim 11, wherein the MEKK polypeptide is human MEKK2.

13. (canceled)

14. The binding motif of claim 11, wherein the amino acid sequence comprises the sequence GRKTFPR.

15. The binding motif of claim 14, wherein the amino acid sequence comprises the sequence YNDGRKTFPRARR.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A method of treating and/or preventing a MEKK-mediated condition in a subject, comprising modulation phosphorylation of a MEKK polypeptide in the subject, wherein the MEKK polypeptide includes the binding motif of claim 1 and phosphorylation of the threonine is modulated.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. The method of claim 32, wherein the MEKK is MEKK2 or MEKK3.

93. The method of claim 32, wherein the subject is a human.

94. (canceled)

95. (canceled)

96. (canceled)

97. (canceled)

98. The method of claim 32, wherein the MEKK-mediated condition is a chronic inflammatory disease or a condition associated with expression of pro-inflammatory cytokines.

99. The method of claim 98, wherein the MEKK-mediated condition is an autoimmune disorder.

100. The method of claim 99, wherein the autoimmune disorder is arthritis, asthma, or lupus.

101. The method of claim 98, wherein the MEKK-mediated condition is arthritis.

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)
